(19) United States
(12) Patent Application Publication
(10) Pub. No.: US 2016/0045276 A1
(43) Pub. Date: Feb. 18, 2016

(54) SYSTEMS AND METHODS FOR IDENTIFYING INSTRUMENTS

(71) Applicant: THE BOARD OF TRUSTEES OF THE UNIVERSITY OF ILLINOIS, Urbana, IL (US)

(72) Inventors: Peter Pfanner, Chicago, IL (US); Matthew Wizinski, Chicago, IL (US); Matthew Clark, Seattle, WA (US); James Hotaling, Salt Lake City, UT (US)

(21) Appl. No.: 14/778,687

(22) PCT Filed: Apr. 4, 2014
(86) PCT No.: PCT/US14/32949

§ 371 Date: Sep. 21, 2015

Related U.S. Application Data
(60) Provisional application No. 61/808,475, filed on Apr. 4, 2013.
(60) Provisional application No. 61/898,583, filed on Nov. 1, 2013.

(51) Int. Cl. A61B 19/00; G06K 7/14; G06F 19/00; A61B 19/02

(52) U.S. Cl. CPC A61B 19/44; A61B 19/02; G06K 7/1443; G06F 19/327; A61B 2019/446

(57) ABSTRACT

A system for identifying contents of an enclosed package. The system includes a camera, a projector, a processor, a display surface, and a plurality of markers. The camera identifies the markers on the packaging of an instrument or group of instruments. The camera is connected to the processor, and the processor is connected to the projector. Once the camera identifies a marker, the processor receives information about the instrument and sends the projector information about the instrument, such as an enlarged view of the instrument or a video illustrating how the instrument may be used. The projector displays the visual information on to the surface.

### RELATED APPLICATIONS

This application claims the benefit of U.S. Provisional Patent Application No. 61/808,475, filed on Apr. 4, 2013, entitled “Systems and Methods for Identifying Instruments,” and U.S. Provisional Patent Application No. 61/898,583, filed on Nov. 1, 2013, entitled “Projection System,” the entire contents of both of which are incorporated herein by reference.

### BACKGROUND

The present invention relates to systems and methods for identifying and visualizing contents of an enclosed package and for providing visual assistance for management of instruments.

Having efficient access to the correct instrumentation is a point of concern during surgery. Smaller instruments may be packaged together in large groups and wrapped in opaque sterile covering, making it difficult to know which package contains the exact instrument needed. Additionally, instruments may look similar to one another with precise distinctions that are difficult to discern. Currently, surgeons and surgical nurses are required to read complicated lists of a package's contents or attempt to discern distinguishing details from diagrams on the package. Often, they will tear open a number of the wrong packages until finding what is needed. By opening and desterilizing the incorrect package, time and materials are wasted. Further, some single use items that can cost thousands of dollars are often not re-useable once opened. Additionally, the surgical staff must account for the exact instruments used during the course of a surgery prior to a surgery's conclusion, which is time consuming. Accordingly, a need exists for systems and methods to improve both the identification process and visibility of the contents of sterilized surgical packages as well as maintain inventory of those instruments used during the course of a surgery as described and claimed herein.

### SUMMARY

Embodiments of the invention relate to systems and methods for identifying and visualizing the contents of sterilized surgical packages, as well as maintaining inventory of instruments used during a surgery. Embodiments of the invention may also be incorporated in other settings such as an assembly line, a factory or manufacturing setting, or a store. The systems and methods may be used in any setting that requires the identification and tracking of a variety of instruments.

One embodiment of the invention provides a system for identifying instruments. The system includes a camera, a projector, a processor, a display surface, and a plurality of markers. The camera identifies the markers on the packaging of an instrument or group of instruments. The camera is connected to the processor, and the processor is connected to the projector. Once the camera identifies a marker, the processor receives information about the instrument and sends the projector information about the instrument, such as an enlarged view of the instrument or a video illustrating how the instrument may be used. The projector displays the visual information on to the surface.

In some embodiments, the invention provides a system including a camera, projector, and processor. The system uses computer vision, image projection, and a labeling system from tracking surgical equipment. For example, the system can use an overhead projector and an HD camera mounted over a table or other surface, to provide tracking and feedback. Both the projector and camera can communicate with a computer. The computer can include computer vision software configured to track fiducial markers.

In some embodiments, the system provides a projection system. The projection system includes a projector that is mounted by means of an attachment mechanism. The projection includes a user interface which may be used to select a desired image to be projected. After the image has been selected, the projector produces a beam of light that creates a pre-determined image on a surface.

The system projects images of the needed instruments onto surgical trays, recognizes and verifies correct instrument selection, and ensures correct placement and setup. Surgeries can be written as recipes to be stored in an online database, with data protections and version control systems in place, but accessible by a supply chain team as well as the surgical team. When a surgical recipe is scheduled in advance, the hospital can insure that the required instruments will be available in inventory on the date of the procedure. On the procedure day, the system can instruct the operator on where the needed items are located within the theater, how they should be prepared, where on the tray they should be placed, in what orientation and what to layout next. The aim is to remove the potential for human error in setup between the surgeon developing a surgical plan and the execution of that plan in theater, repeatedly, with confidence.

By developing a coordinated system of storage hardware, software and object recognition units, human error can be removed from the process of surgical preparation by guiding the user to locate, place, setup, track and inventory instruments within the surgical theater. By developing a coordinated system of storage hardware, software and object recognition units, surgeons can prepare, track, refine and share their procedures. By developing a coordinated system of storage hardware, software and object recognition units, hospitals can more accurately track and eliminate systemic waste and inefficiency in inventory management by having better data access to surgical requirements and real-time inventory levels throughout the premises or network.

Other aspects of the invention will become apparent by consideration of the detailed description and accompanying drawings.

### BRIEF DESCRIPTION OF THE DRAWINGS

FIG. 1 is a block diagram of one embodiment of a system for displaying information about the contents of a package.

FIG. 2 is a perspective view of the system of FIG. 1.

FIG. 3 is a flowchart of a method for displaying information based on the location of a marker using the system of FIG. 1.

FIG. 4 is a flowchart of another method of using the system of FIG. 1 to display information about the contents of a package.

FIG. 5 is an overhead view of a display surface of the system of FIG. 2 with a marker positioned in an area outside of a hotspot.

FIG. 6 is an overhead view of the display surface of FIG. 5 with the marker positioned inside a hotspot.

FIG. 7 is a flowchart illustrating a method for identifying instruments using the system of FIG. 2.

FIG. 8 is a perspective view of a system for tracking tools used during a procedure.

FIG. 9 is a flowchart of a method of tracking tools used during a procedure using the system of FIG. 8.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

Also, it is to be understood that the phraseology and terminology used herein is for the purpose of description and should not be regarded as limited. The use of “including,” “comprising” or “having” and variations thereof herein is meant to encompass the items listed thereafter and equivalents thereof as well as additional items. The terms “mounted,” “connected” and “coupled” are used broadly and encompass both direct and indirect mounting, connecting and coupling. Further, “connected” and “coupled” are not restricted to physical or mechanical connections or couplings, and can include electrical connections or couplings, whether direct or indirect. Also, electronic communications and notifications may be performed using any known means including direct connections, wireless connections, etc.

It should be noted that a plurality of hardware and software based devices, as well as a plurality of different structural components may be utilized to implement the invention. Furthermore, and as described in subsequent paragraphs, the specific configurations illustrated in the drawings are intended to exemplify embodiments of the invention and that other alternative configurations are possible.

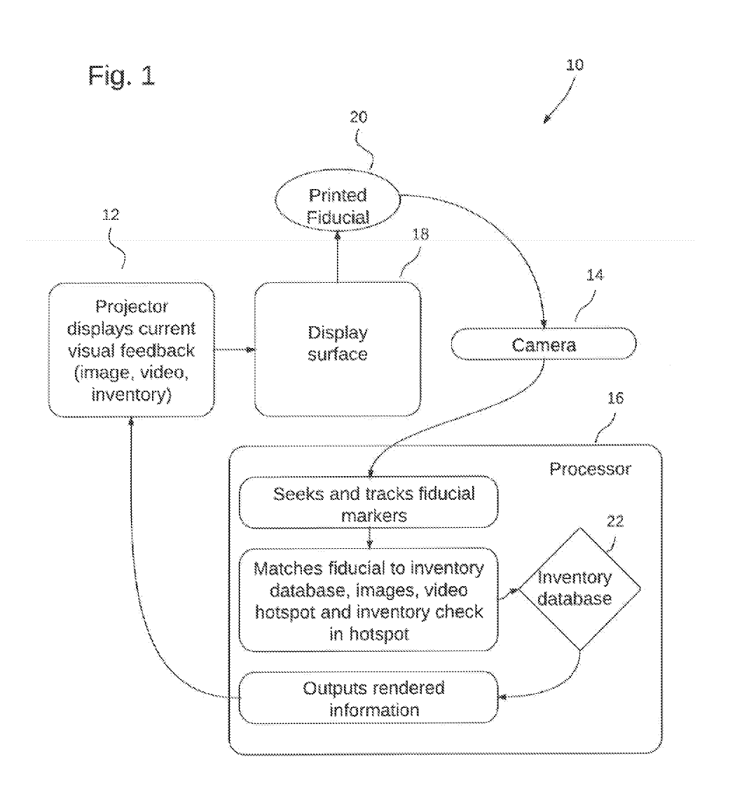

FIG. 1 schematically illustrates a system 10 according to one embodiment of the invention. As shown in FIG. 1, the system 10 includes a projector 12, a camera 14, a processor 16, a display surface 18, and a marker 20 (e.g., a “printed fiducial”). The processor 16 may include a database 22 that may store an inventory of possible instruments or instruments used with the system 10. The database 22 can be stored to a non-transitory computer-readable memory of the system 10, for example. As shown in FIG. 1, the camera 14 communicates with the processor 16. The processor 16 also communicates with the projector 12.

The camera 14 identifies fiducial markers 20 on the packaging of an instrument. Fiducial markers are unique visual symbols representing numeric data. As the camera images and processes the fiducial markers, the markers and their locations are tracked by the computer vision software. The markers may be printed on paper labels and affixed to the surgical equipment packaging. When an instrument package having a fiducial marker is placed on the table, the camera detects the fiducial marker and, based on the information provided by the fiducial marker, identifies instruments in the package. The projector is configured to generate an image of the package's contents and project the image onto the table (e.g., beside the actual package). Accordingly, the act of placing a package on the table such that the camera can read the fiducial marker triggers immediate visual feedback, which eliminates the need to open the package or to read a list of the package's contents. For displayed instruments having small or difficult to decipher features, a photographic detail of the feature(s) can be projected with the overall image of the instrument, either as a static photograph or video.

When an instrument package having the fiducial marker 20 is placed on the display surface 18, the camera 14 sends an image of the fiducial marker 20 to the processor 16 where it is identified. The processor 16 also determines information related to the location of the fiducial marker 20 on the display surface. Once the fiducial marker 20 is identified, the processor 16 sends information to the projector 12 based on the data encoded in the fiducial marker 20 and the location information of the package having the marker 20. Such information can include image or video information (e.g., an enlarged photograph of the instrument, a video demonstrating use of the instrument, etc.) that the projector 12 then projects onto the display surface 18. It should be noted that the display surface 18 may be a table, a wall, or any other surface where an image may be viewed. However, in some constructions, the display image may instead be transmitted directly to a viewing screen (e.g., a computer monitor, television screen, etc.), which may or may not require the use of the projector 12.

The display surface 18 includes a main area and multiple hotspots. When a marker 20 is identified by the camera 14 in the main area, an image of the instruments contained within the package related to the marker 20 is displayed. In some systems, hotspots are predefined areas within the main area of the display surface 18 that the marker 20 can be placed within, as described in further detail below. Placing the marker 20 within a hotspot can trigger different outputs from the processor 16. A hotspot can be dedicated to revealing further information about an instrument package beyond displaying an image of its contents. Hotspots can also perform particular actions based on the marker 20. For example, placing the marker 20 within a video hotspot can display a video demonstrating use of a particular instrument in the package. Further, an inventory hotspot can be dedicated to inventory and tracking purposes.

For example, a package about to be opened for use can be checked into the inventory database 22 by placing the marker 20 for that package in the inventory hotspot. Checking the package into the inventory database 22 can include adding data related to the contents of the package into the inventory database 22. For example, the data can be used by the processor 16 to maintain a record of active instruments for that particular surgery, as well as a record of opened packages that must be accounted for before the surgery's conclusion. The record of active instruments can include location information for various instruments throughout the surgery. This location information can include, for example, the location of related tools within the operating room by drawer or shelf number. Furthermore, as described in further detail below, the projector can display an image of a tool that has been opened during the current procedure on the surface such that the user can place the tool at the recommended location when it is not in use. Alternatively or additionally, the record of active instruments can be communicated with other existing computer or database systems. The inventory database 22, as well as the record of active instruments, can also be integrated into existing hospital systems, such as to maintain supply inventories and for cost and billing purposes. It should be noted that actions triggered by any of the video, inventory, or other hotspots can be performed either as an alternative to or in addition to displaying the images of a package's contents, as will be described below in further detail.

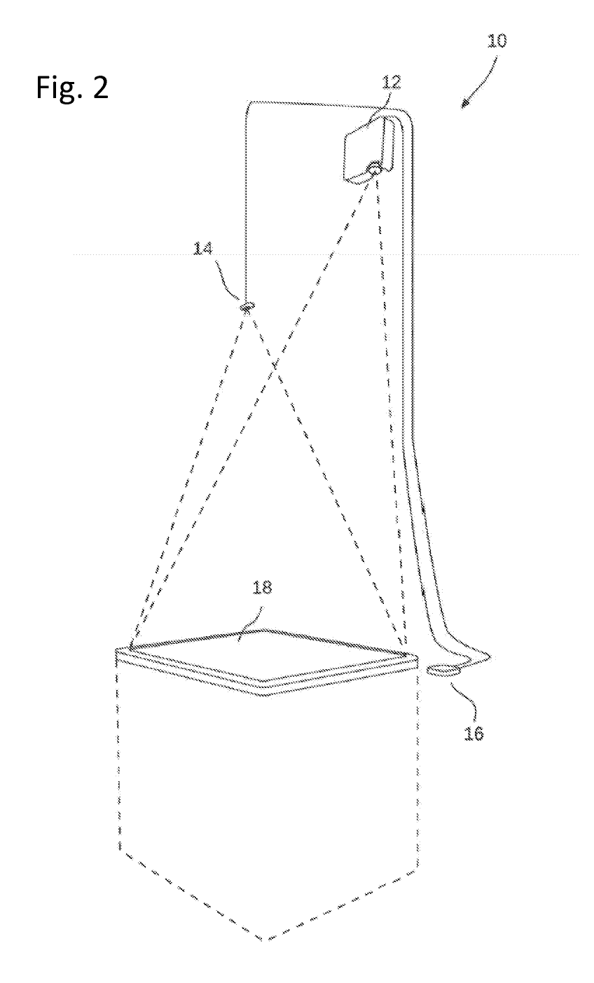

FIG. 2 illustrates a possible configuration of the system 10 according to one embodiment. In FIG. 2, the camera 14 is placed directly over a surface 18. When an instrument or package is placed within the field of view of the camera 14, the camera 14 detects the marker 20 and transmits information about the marker 20 to the processor 16. Once the marker is identified, the processor 16 outputs information to the projector 12. The projector 12 then transmits various images to the surface 18.

Although, in the system of FIG. 2, the images are projected on the same surface as the area for reading the marker, in some constructions, multiple surfaces can be used. For example, an area for reading the marker 20 can be separate from an area for projecting any media (e.g., images, videos, etc.) related to the marker 20. In keeping the marker reading and media projection areas separate, more users can benefit from the system 10 at once. For example, a nurse who is handling an instrument can scan the marker 20 of the package, while the image can be projected to an area where a surgeon can more easily view the contents of the package without having to move from his position. Furthermore, the media related to the marker 20 can be projected on the surface 18 as well as one or more additional displays (e.g., monitors).

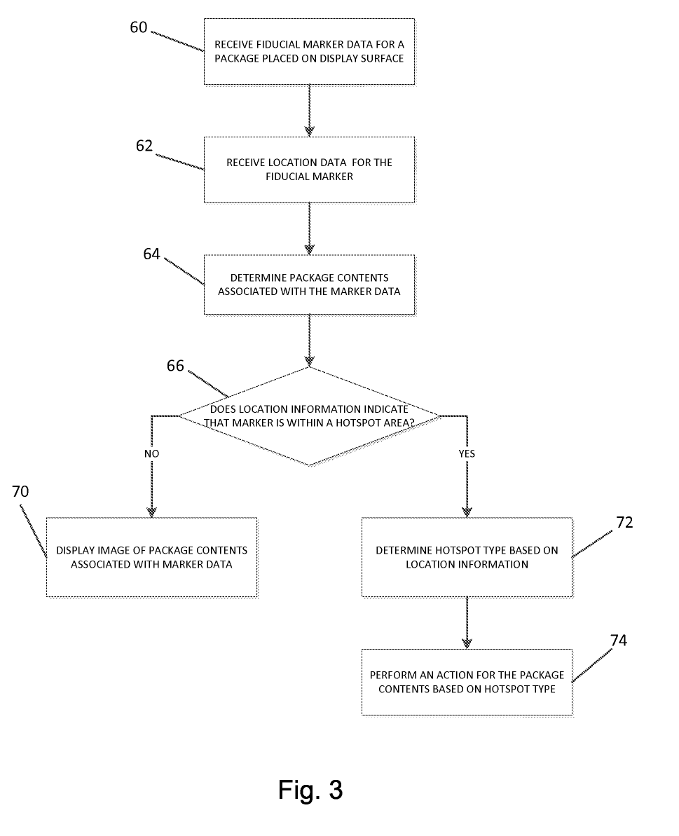

FIG. 3 illustrates a method for using the system of FIG. 2 to identify and manage the contents of a package. When a package is placed on the display surface 18, the processor 16 receives data associated with the marker 20 via the camera 14 (step 60). The received data associated with the marker 20 can include, for example, identification codes associated with each item contained in the package, as well as any additional information associated with each identification code or the package itself. The processor 16 also receives location data associated with the marker 20 via the camera 14 (step 62). The received location data can include, for example, coordinates or other locational identifiers associated with the position of the marker 20 on the display surface 18. Based on the received marker data, the processor 16 determines the package contents (step 64). For example, the processor 16 may access a look-up table or inventory database 22 to match the received marker data with corresponding instruments. It should be noted that the processor 16 can be configured to access other databases either stored locally to a non-transitory computer-readable memory of the system 10, or externally to other hardware systems and server networks (including, for example, remote databases accessible via the Internet). Determining the package contents allows the processor 16 to locate various stored information or media associated with the package contents, such as videos, read/write locations for inventory purposes, images, etc.

Based on the received location information, the processor 16 determines whether the marker 20 is located within a hotspot region (step 66). For example, the processor 16 may be able to access a map of the display surface 18 (e.g., from a memory module in communication with the processor 16), which can be subdivided into various regions including the main area and multiple hotspots. If the received location information suggests that the marker's 20 location falls within the main area but not a hotspot region, the processor 16 prompts the projector 12 to display image data of the package contents on the display surface 18, based on the received marker data (step 70). However, if the received location information suggests that the marker 20 is located within a hotspot region, the processor 16 determines the hotspot type (e.g., video hotspot, inventory hotspot, etc.) (step 72). For example, the processor 16 can determine the hotspot type based on whether the specific locational identifier of the marker 20 falls within a group of locational identifier values encompassed by a particular hotspot region. When the processor 16 determines the hotspot type, the processor 16 performs the predefined action associated with the hotspot type for the package contents (step 74). For example, if the processor 16 determines that the marker 20 is within the inventory hotspot, the processor 16 executes control logic for storing information related to the package contents in the inventory database 22.

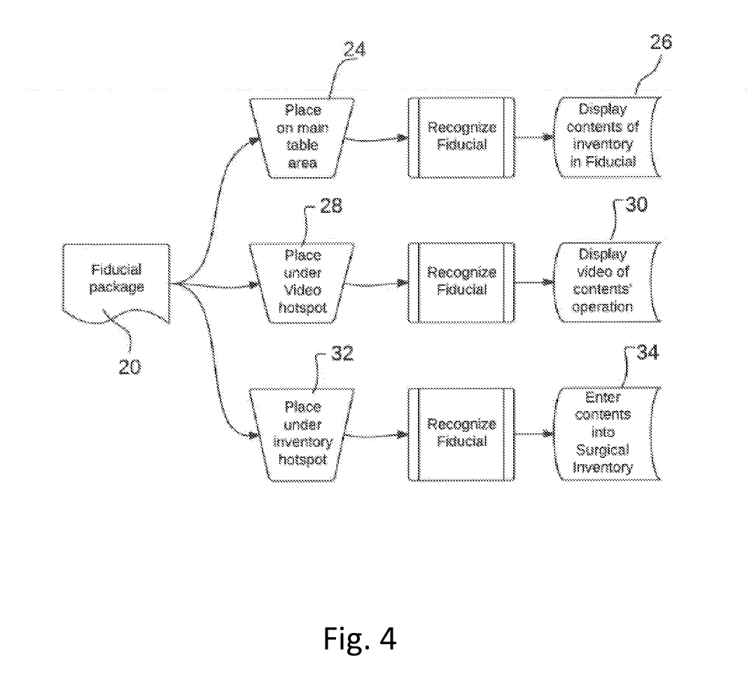

FIG. 4 illustrates how the marker 20 may trigger various actions depending on the marker's location on the display surface 18. In some constructions, the system 10 can visibly (to a user) track the location of the marker 20 in the display area 18. For example, the system 10 can illuminate the marker 20 using a spotlight generated by the projector 12. Further, the system 10 can be configured to follow the marker 20 with the spotlight as the marker 20 is moved within the display area 18. It should be noted, however, that the system 10 can also be configured to visibly track the marker 20 by other means, such as with a laser projection, for example.

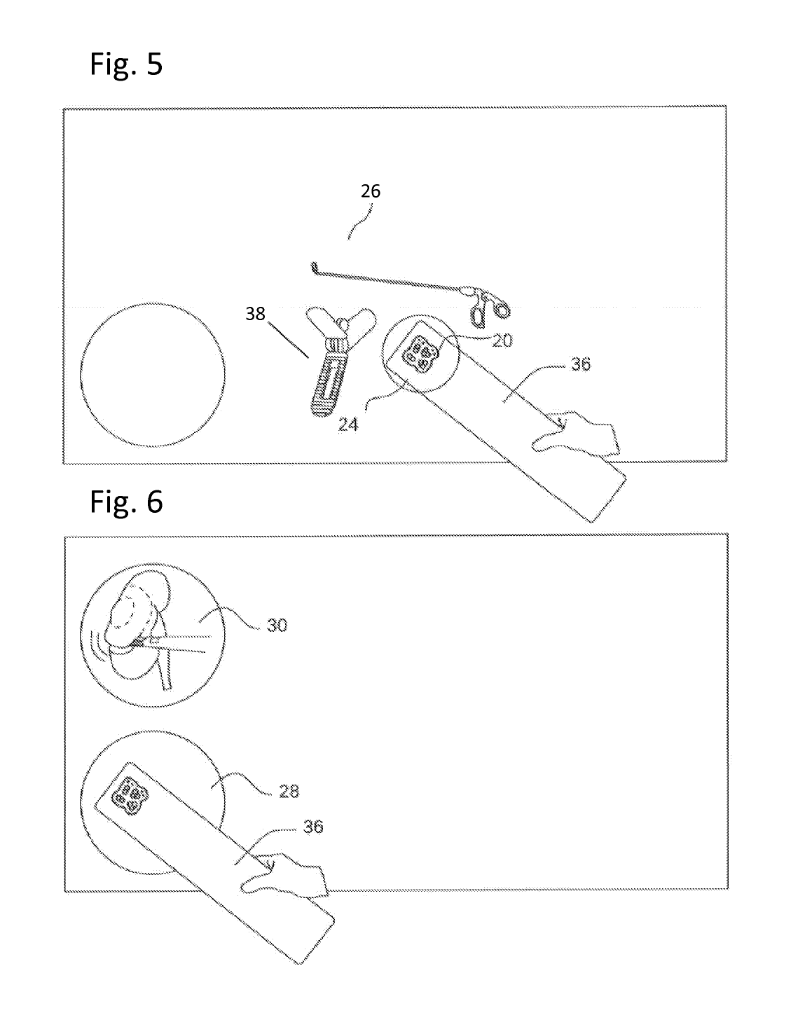

As described above, the display surface 18 can include a main area 24, as well as various hotspots within the main area 24 including a video hotspot 28 and an inventory hotspot 32. If the marker 20 is placed in the main area 24, the processor 16 signals the projector 12 to display an image 26 of the package contents associated with the marker 20. For example, FIG. 5 illustrates a package 36 being placed on the display area 18 such that the marker 20 can be identified within the main area 24.

As shown in FIG. 5, the image 26 is displayed beside the marker 20. In some embodiments, the image 26 can be displayed at a fixed location on the display surface 18, not necessarily beside the location of the marker 20. However, in other embodiments, the image 26 can be made to follow the marker 20 within a certain distance of the marker 20. For example, the image 26 can be displaced as the marker 20 is displaced, and in such a way that the image 26 is always displayed within a predefined distance of the marker 20 (i.e., “follows” within a predefined distance of the marker 20 as the marker 20 is moved across the surface 18). Alternatively, the distance between the projected image 26 and the marker 20 can be variable based on the location of the marker 20. As also shown in FIG. 5, an enlarged view 38 of the image 26 can also be displayed for instruments having complex features, or features that are too small to discern from the image 26. In the case of FIG. 5, the enlarged view 38 displays a detailed view of the small component on the tip of the instrument shown in the image 26.

As shown in FIG. 4, if the marker 20 is placed within the video hotspot 28, the projector 12 displays a video 30 demonstrating the use of an instrument associated with the marker 20. However, it should be noted that placing the marker 20 within the video hotspot 28 can cause the projector 12 to display videos other than demonstrative videos. Referring to FIG. 6, placing the marker 20 within the video hotspot 28 causes the projector 12 to display the video 30 beside the video hotspot 28. However, as described above with respect to FIG. 5 (for the image 26), the video 30 can be displayed at a fixed location on the display surface 18 that is not necessarily directly beside (or within a certain distance of) the video hotspot 28. Finally, as shown in FIG. 4, placing the marker 20 within the inventory hotspot 32 causes the processor 16 to store the contents of the package associated with the marker 20 into a surgical inventory, or record of active instruments 24 (as previously described with respect to FIG. 1).

The examples presented above provide only a few types of hotspots that can be implemented by the system. Other additional hotspots can be implemented in other constructions of the system 10. Furthermore, in some constructions, multiple hotspots or the functions of multiple hotspots can be combined. For example, one hotspot can cause the projector 12 to display both an exploded view of an instrument, as well as a video demonstrating the use of that instrument. Another construction may include a hotspot that causes the processor to enter an instrument into the inventory database 22, as well as cause the projector 12 to display an image of the instrument.

Although the examples presented above focus on the context of a surgical operating room, the systems described herein can be implemented in other situations as well. For example, the system 10 can be implemented for use in assembly lines, factory or manufacturing settings, and stores. Accordingly, the system 10 can be implemented in any setting that requires the identification and tracking of a variety of instruments. For instance, on a moving assembly line, inventoried packaged parts are delivered to individual assembly stations along the line, such as a moving automobile assembly line. In such a setting, the packaged parts can include the fiducial marker 20. Further, viewing stations comprised of the camera 14 and the projector 12 can be set up at each assembly station along the assembly line. At the viewing stations, workers can place a packaged part on the display surface 18 to view the package contents, as well as other information such as assembly instructions, for example.

An example of additional information that can be displayed with the viewing contents can be a specific amount of force (e.g., a maximum force, etc.) that can be applied to the package contents during assembly. Also, any Occupational Safety and Health Administration (OSHA) guidelines for working with specific instruments contained in the package can be displayed to reinforce workplace safety. This can be done, for example, by designating a hotspot as a safety hotspot by which a worker can view the OSHA guidelines by placing the marker 20 inside the safety hotspot. As mentioned above, any and all hotspots may be tailored to the particular needs of the environment where the system 10 may be used. Accordingly, in some embodiments, the hotspots can be configured to perform customized actions.

In an exemplary food manufacturing setting, packaged ingredients may be placed on the display surface 18 to view a list of composite ingredients and requirements for that particular viewing station. Also, any safety measures may be displayed in a safety hotspot, as described above for the assembly line setting.

In high precision manufacturing, security industries, and related industries, the marker 20 can be used to track which workers had access to the package. For example, in one construction, every time the marker 20 is logged or checked-into a hotspot (similar in functionality to the inventory hotspot described above), its status can be amended in the database 22 as having been logged at a particular station by a certain person at a specified time.

The marker 20 can also be used to for access control applications. For example, access to certain packages bearing the marker 20 can be limited by security measures (e.g., physical measures, password protected measures, etc.) so that the marker 20 can be encoded with information about who is allowed access to the package. Such encoded or tagged information can include an employee ID, an image, a retinal scan a fingerprint scan, etc.

The system 10 can also be used in a retail environment. In particular, a customer can select a package bearing the mark 20 (e.g., a box of toys) and place the package under the camera 14. The package contents and/or additional information can then be displayed for the customer, either via a display screen, a projected image, etc. The system 10 can also be incorporated into currently existing technology, such as retail store price scanners. In some cases, the system 10 would then be able to access databases or other storage locations associated with the existing technology. Accordingly, the system 10 can have a broader access to perform diverse functionality.

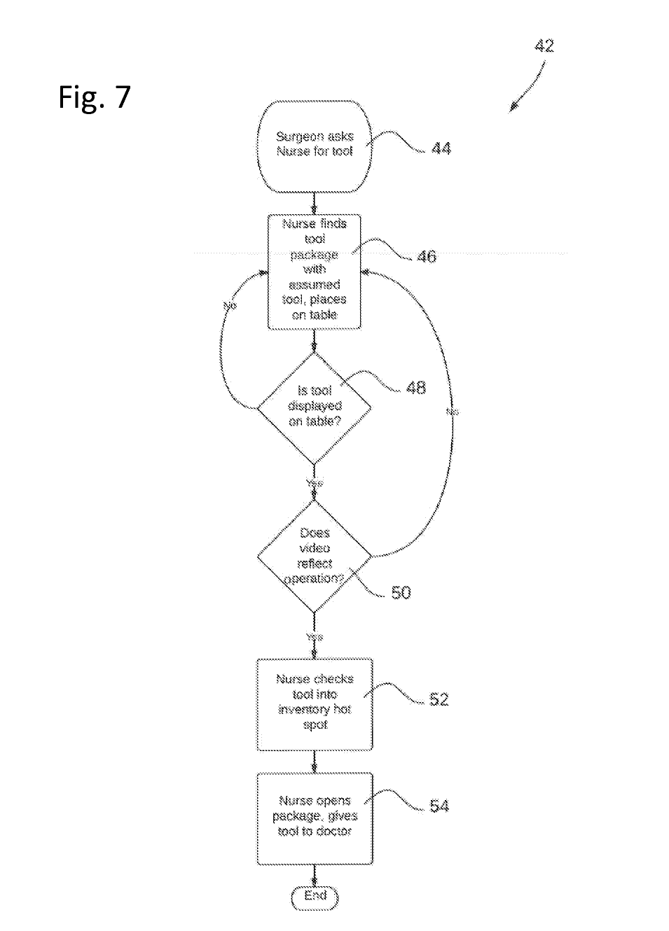

FIG. 7 is a flowchart illustrating an exemplary method 42 for using the system 10 in a specific scenario related to surgery. The method 42 includes identifying a marker, enabling a variety of hotspots, and entering an instrument into the inventory. In particular, a surgeon can ask a nurse for a specific instrument (step 42). The nurse then locates a package bearing the marker 20 assumed to contain the instrument, and places the package in the main area 24 of the surface 18 (step 46). The nurse can then view if the requested instrument is inside the package by determining whether an image of the instrument is displayed on the surface 18 (step 48). If the image is not displayed to the surface 18, then the nurse can locate a new package to place on the table (step 46). However, if the image of the requested instrument is displayed on the surface 18, the nurse can place the marker 20 within the video hotspot, such that a video demonstrating the use of the instrument can be displayed to the surface 18. The nurse can then determine if the identified instrument can be used for the desired operation (step 50). If the video does not reflect the desired operation or use of the instrument, the nurse can locate a new package to place on the table (step 46). However, if the video does reflect the desired operation, the nurse can check the instrument into the inventory database 22 by placing the marker 20 within the inventory hotspot 34 (step 52). After checking the instrument into the database 22, the package can then be opened and the requested instrument provided to the surgeon (step 54).

It should be noted that the method 42 can be performed with additional steps or alternative orders, or, further, with different hotspots. For example, a user can use the video hotspot to view a demonstration of an operation, as opposed to simply viewing an image of the instrument itself. Just as the system 10 can be used in different settings, the method 42 can be used in other settings, as well, such as the alternative settings described above. The method 42 and system 10 can be varied depending on the need of the particular environment.

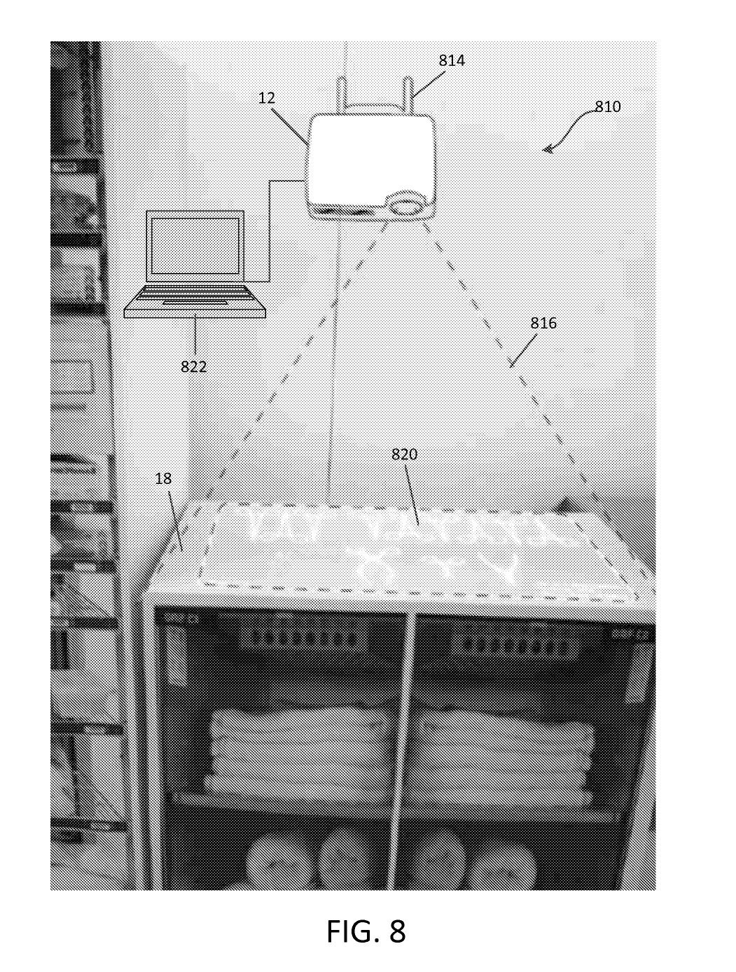

FIG. 8 illustrates another example of a projection system 810. In the example of FIG. 8, the projection system 810 includes the projector 12, the display surface 18, an attachment mechanism 814, and a user interface 822. The projector 12 emits a light beam 816 that causes an image 820 to be displayed on to the display surface 18. In some constructions, the display surface 18 may be a surface located in an operating room, thereby assisting health care personnel and others that assist both before, during, and after a procedure performed in an operating room. Through the user interface 822, a user selects a procedure to be performed, such that the system 810 can display the appropriate corresponding information. The user interface 822 is shown in the example of FIG. 8 as a personal computer. However, the user interface 822 can be implemented as an interface integrated into the projector 12, a separate stand-alone interface, or a networked connection to a device at a remote location (such as a computer located in a surgeon's office).

The projection system 810 also includes the inventory database 22 and a power source (not shown). As previously described, the inventory database 22 stores data needed to produce lists of instruments for surgeries and images of the instruments themselves. The projection system 810 also includes a camera, such that additional instruments can be entered into the system by placing the instrument on the display surface 18 and capturing an image of the instrument to be stored to the inventory database 22. As noted above, the inventory database 22 can be stored locally on a non-transient, computer readable memory within the projection system 810. However, in other constructions, the inventory database 22 is stored on a non-transient, computer-readable memory located at a remote server that is accessible through a network connected (e.g, an Internet connection).

Once a procedure has been selected through the user interface 822, the projection system 810 projects the image 820 of instruments necessary for a procedure onto the display surface 18. The camera integrated into the projector 12 of the projection system 810 allows the projection system to recognize and verify correct instrument selection, placement, and setup as the physical instruments are placed on the surface 18. For example, surgical procedures can be written as a series of steps (e.g., “recipes”) stored in the inventory database 22 or other data sources, and can be accessible by a supply chain team as well as the surgical team. When a surgical procedure is scheduled in advance, the hospital can insure that the required instruments will be available in inventory on the date of the procedure. This also ensures that all of the procedures are consistent and organized.

On the day of the procedure, the projection system 810 displays an image on the surface 18 instructing the operator on where the needed items are located within the operating room, how they should be prepared, where on the display surface 18 they should be placed, in what orientation, and what to layout next. Accordingly, the projected image 820 can depict the exact layout of the instruments needed, in order in when they will be needed during a procedure. During a procedure, the projection system 810 uses recognition software to track instruments, so that the system 810 will know which instrument will be used next in the procedure. Furthermore, the recognition software allows the system 810 to determine which instrument(s) from the list of required instruments is missing from the surface 18 (e.g., which instruments are currently in use or have not be returned to the surface 18 after use). The mechanism reduces the potential for human error in setup between the surgeon developing a surgical plan and the execution of that plan in operating room theater, repeatedly, with confidence.

The projection system 810 also includes the capability to add additional instruments that may be needed during surgery. For example, if an extra instrument that was not included in the original recipe is brought in to the surgery, the projection system 810 may photograph and add the new instrument to the inventory database 22 to be added to the projected image 820 and the surgical recipe. In some constructions, an instrument to be added during the surgery or other procedure can be placed in the inventory hotspot such that the instrument can be added to the inventory database 22. Therefore, the projection system 810 may capture and update data dynamically in the inventory database 22 or a non-transitory computer-readable memory of the system 810.

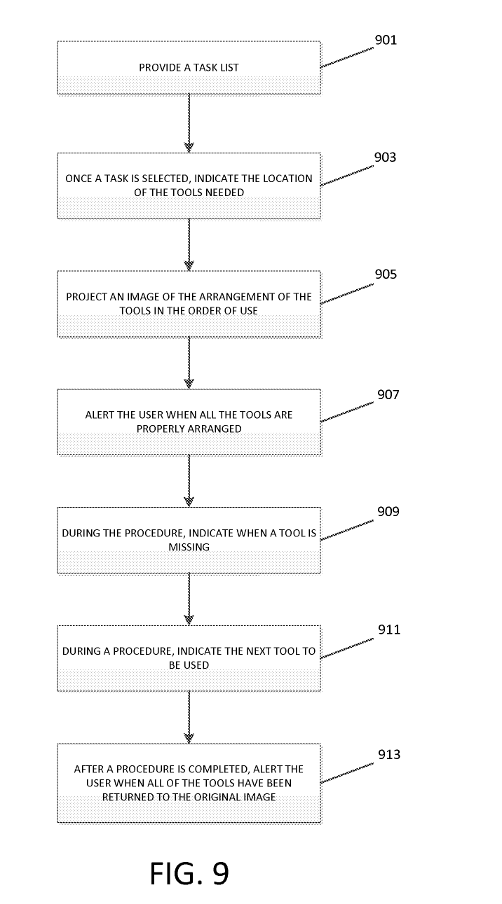

FIG. 9 illustrates a method of using the projection system 810 during a surgical procedure. First, the projection system 810 displays a task list to the user (either on the surface 18 or through the user interface 822 (step 901). For example, the projection system 810 may present the user with a schedule of procedures to be performed, and the user may select which procedure to prepare necessary instruments for. Once a task is selected, the projection system 810 indicates the location of each of the instruments needed for the procedure (step 903). For example, the projection system 810 may provide the user with a list of instruments and indicate where the user may find each instrument in the operating room. The projection system 810 may be as specific as indicating which drawer or shelf in which to find the tool needed.

The projection system 810 projects an image of the arrangement of tools needed for the selected procedure onto the surface 18, and can present the arrangement of tools in the order of use for each tool (step 905). As described above, the image will mimic the actual structure of the tool, so it may help the user place the tools in the corresponding spaces quickly and easily. The projection system 810 also alerts the user when all of the tools for the procedure are properly arranged (step 907). For example, the projection system 810 may make a certain noise, the color of the light beam 816 may change, or a word or symbol may be displayed when all of the tools are arranged in their corresponding locations.

As described above, the projection system 810 is configured to monitor the physical placement of tools on the surface 18. Using this information, the projection system 810 also alerts a user during the procedure when a tool is missing from the surface (step 909). For example, when a tool is removed from the surface, the image of that tool is highlighted in a different color, thus indicating that no tool is present in that corresponding spot. The projection system 810 also indicates which instrument is going to be needed for the next step in the procedure (step 911). For example, the projection system 810 may highlight the next instrument by highlighting the instrument depicted in the projected image. However, in other constructions, the projection system 810 may indicate which instrument to use next by displaying the name or picture of the tool on the user interface 822.

After a procedure is completed, the projection system 810 alerts a user when all of the tools have been returned to the surface (step 913). This final step will ensure that no tools are missing at the end of the procedure. In some constructions, the method may be altered or changed in a way to better suit the user or to carry out the procedure efficiently and effectively.

Thus, embodiments of the invention provide, among other things, systems and methods for identifying and visualizing contents of an enclosed package. Various features of the invention are set forth in the following claims.

What is claimed is:

1. A system for providing information related to contents of a package, the system comprising:
an optical sensor configured to read a marker on a package placed on a surface;
a projector configured to display image data on the surface;
a processor coupled to the optical sensor and the projector; and
a memory storing instructions that, when executed by the processor, cause the system to:
determine whether a package bearing a marker is placed on the surface based on data captured by the optical sensor,
identify the contents of the package based on data relating to the marker captured by the optical sensor, and
project an image on the surface based on the identification of the contents of the package.

2. The system of claim 1, wherein the surface includes a hotspot region, and wherein the instructions, when executed by the processor, further cause the system to:
determine a location of the marker on the surface based on the data captured by the optical sensor;
determine, based on the location of the marker on the surface, whether the marker is positioned in the hotspot region; and
perform a predefined action associated with the hotspot region when the marker is determined to be positioned in the hotspot region.

3. The system of claim 2, wherein the instructions, when executed by the processor, cause the system to perform the predefined action associated with the hotspot region by
projecting video data on the surface when the marker is determined to be positioned in the hotspot region, the video data relating to at least one object contained in the package, and
wherein the instructions, when executed by the processor, cause the system to project the image on the surface based on the identification of the contents of the package by
projecting a still image of at least one object contained in the package on the surface when the marker is determined to be positioned outside of the hotspot region on the surface.

4. The system of claim 3, wherein the video data includes a video demonstrating how the contents of the package are properly used.

5. The system of claim 2, wherein the instructions, when executed by the processor, cause the system to perform the predefined action associated with the hotspot region by
storing information related to the contents of the package to an inventory stored on the memory.

6. The system of claim 1, wherein the optical sensor includes a camera configured to capture image data of objects placed on the surface.

7. The system of claim 6, wherein the instructions, when executed by the processor, cause the system to receive an image of the marker from the camera and to identify the marker based on the image from the camera.

8. The system of claim 1, wherein the instructions, when executed by the processor, cause the system to identify the contents of the package by matching information from the marker with a record stored in a data source.

9. The system of claim 8, wherein the record stored in the data source further includes a list of objects contained in a package bearing the corresponding marker.

10. The system of claim 1, wherein the instructions, when executed by the processor, further cause the system to
identify a location of the marker on the surface, and
project an image on the surface proximate to the location of the marker on the surface.

11. The system of claim 10, wherein the instructions, when executed by the processor, further cause the system to:
determine when the location of the marker has changed, and
change the location of the projected image based on the changed location of the marker.

12. The system of claim 1, wherein the instructions, when executed by the processor, further cause the system to:
receive a selection of a procedure to be performed; and
project a still image to the surface, the still image including an arrangement of at least one object to be used during the procedure.

13. The system of claim 12, wherein the instructions, when executed by the processor, further cause the system to determine when a physical object has been placed on the surface proximate to a corresponding projected image of the object.

14. The system of claim 13, wherein the instructions, when executed by the processor, further cause the system to indicate when the physical object is not placed on the surface proximate to the corresponding projected image of the object.

15. The system of claim 12, wherein the still image projected on the surface includes a plurality of objects to be used during the procedure arranged in an order in which the objects are to be used during the procedure.

16. The system of claim 15, wherein the instructions, when executed by the processor, further cause the system to indicate which object should be used next based on the order in which the objects are used during the procedure.

17. The system of claim 12, wherein the instructions, when executed by the processor, further cause the system to determine, at an end of the procedure, whether the at least one object is placed on the surface proximate to the corresponding projected image of the object.

18. A method for identifying contents of a package, the method comprising:
capturing, by an optical sensor, an image of a marker affixed to a package placed on a surface;
identifying, by a processor, contents of the package based on the image of the marker captured by the optical sensor; and
displaying an image of the contents of the package on the surface.

19. The method of claim 18, further comprising;
determining, by a processor, a location of the marker on the surface based on the image captured by the optical sensor;
determining, by a processor and based on the location of the marker on the surface, whether the marker is positioned in a hotspot region of the surface; and
performing, by a processor, a predefined action associated with the hotspot region when the marker is determined to be positioned in the hotspot region.

20. The method of claim 19, wherein performing the predefined action includes;
projecting, by a processor, video data on the surface when the marker is determined to be positioned in the hotspot region, the video data relating to at least one object contained in the package, and
projecting, by a processor and based on identifying the contents of the package, a still image of at least one object contained in the package on the surface when the marker is determined to be positioned outside of the hotspot region on the surface.

21. The method of claim 20, wherein projecting the video data includes projecting a video demonstrating how the contents of the package are properly used.

22. The method of claim 19, wherein performing the predefined action further includes storing information related to the contents of the package to an inventory stored on a non-transitory computer-readable memory.

23. The method of claim 18, wherein capturing the image with an optical sensor includes capturing the image with a camera configured to capture image data of objects placed on the surface.

24. The method of claim 23, further including receiving the image of the marker from the camera and identifying the marker based on the image from the camera.

25. The method of claim 18, further including identifying the contents of the package by matching information from the marker with a record stored in a data source.

26. The method of claim 25, wherein matching the information from the marker with the record stored in the data source further includes matching the information from the marker with a list of objects contained in a package bearing the corresponding marker.

27. The method of claim 18, further including;
identifying, by a processor, a location of the marker on the surface, and
projecting, by a processor, an image on the surface proximate to the location of the marker on the surface.

28. The method of claim 27, further including;
determining, by a processor, when the location of the marker has changed, and
changing, by a processor, the location of the projected image based on the changed location of the marker.

29. The method of claim 18, further including;
receiving a selection of a procedure to be performed; and
projecting a still image to the surface, the still image including an arrangement of at least one object to be used during the procedure.

30. The method of claim 29, further including determining when a physical object has been placed on the surface proximate to a corresponding projected image of the object.

31. The method of claim 30, further including indicating when the physical object is not placed on the surface proximate to the corresponding projected image of the object.

32. The method of claim 29, wherein projecting the still image on the surface includes projecting a still image of a plurality of objects to be used during the procedure arranged in an order in which the objects are to be used during the procedure.

33. The method of claim 32, further including indicating which object should be used next based on the order in which the objects are used during the procedure.

34. The method of claim 29, further including determining, at an end of the procedure, whether the at least one object is placed on the surface proximate to the corresponding projected image of the object.
